(19) United States
(12) Patent Application Publication
(10) Pub. No.: US 2025/0229092 A1
(43) Pub. Date: Jul. 17, 2025

(54) NEUROSTIMULATION WITH ENHANCED PASSIVE RECOVERY

(71) Applicant: Boston Scientific Neuromodulation Corporation, Valencia, CA (US)

(72) Inventor: Travis McCoy, Slidell, LA (US)

(21) Appl. No.: 19/009,319

(22) Filed: Jan. 3, 2025

Related U.S. Application Data
(60) Provisional application No. 63/620,647, filed on Jan. 12, 2024.

(51) Int. Cl. A61N 1/372; A61N 1/05; A61N 1/36

(52) U.S. Cl. CPC A61N 1/37247; A61N 1/0534; A61N 1/3605; A61N 1/36132; A61N 1/37264

(57) ABSTRACT

A system may include a neurostimulator and a programmer. The neurostimulator may have a plurality of electrodes, and may be configured to deliver neurostimulation by delivering neurostimulation pulses of a first polarity to a neural target using at least one stimulation electrode from the plurality of electrodes and passively recovering charge using a first number of one or more passive electrodes from the plurality of electrodes. The programmer may have a user interface configured to receive a user input for changing a number of passive electrodes used to passively recover charge from the first number to a second number. The programmer may be configured to control the neurostimulator to passively recover charge using the second number of passive electrodes. Some examples may include fractionalization to control the contribution of each passive electrode used to recover charge. A variable impedance may be used to control a relative charge recovery current.

### CLAIM OF PRIORITY

This application claims the benefit of U.S. Provisional Application No. 63/620,647, filed on Jan. 12, 2024, which is hereby incorporated by reference in its entirety.

### TECHNICAL FIELD

This document relates generally to medical systems, and more particularly, but not by way of limitation, to systems, devices, and methods for enhancing passive charge recovery for electrostimulation such as neurostimulation.

### BACKGROUND

Medical devices may include devices configured to deliver a therapy to a patient. For example, these devices may include wearable devices and implantable devices. A wearable device, by way of example and not limitation, may include a transcutaneous electrical nerve stimulation (TENS) device. Some implantable devices may use one or more leads to sense electrical signals or to treat various biological disorders, such spinal cord stimulators (SCS) to treat chronic pain, cortical and Deep Brain Stimulators (DBS) to treat motor and psychological disorders, Peripheral Nerve Stimulation (PNS) including Vagus Nerve Stimulation (VNS), Functional Electrical Stimulation (FES), and other neural stimulators to treat urinary incontinence, sleep apnea, shoulder subluxation, and the like. Some of these devices may be connected to an electrode array having a plurality of electrodes that can be selectively activated for use to deliver neurostimulation to a targeted region while attempting to avoid side effects that may be caused by stimulation of untargeted regions.

For example, DBS uses an array of electrodes implanted in the brain. Movement disorder patients experience progression in their disease state as they age. DBS patients may also suffer from this progression, which reduces the efficacy of the stimulation over time and reduces the therapeutic window, which results in unwanted side effects as stimulation is increased in attempt to recapture therapy efficacy.

Some neurostimulation systems may implement active recharge by actively driving both phases of a neurostimulation pulse. For example, some may implement active recharge when neurostimulation is delivered at higher neurostimulation frequencies such as 150 Hz or above. Active recharge reverses the polarity of the electrodes to get the tissue back to a resting state (e.g., 70 millivolts). Such systems may remove charge that can cause side effects, but these active recharges systems use about twice as much power as a system that uses passive charge recovery. Passive charge recovery also has challenges. When there is higher frequency, some electrical charges are left as they are not being removed fast enough. However, when a lower frequency stimulation is used, the stimulation pulse may be wider, which also can mean a larger charge is being left.

Both multiple independent current control (MICC), which independently controls the current sourced or sunk at each active electrode, and directional DBS leads which use segmented electrodes around a periphery of the lead, helps with avoiding unwanted stimulation in areas of the brain that create side effects. However, although directional leads improve therapeutic outcomes, these directional leads also make it more difficult to passively recover charge introduced by the neurostimulation therapy. Conventionally, only the electrodes that are activated to deliver the neurostimulation are used to passively recover charge, and the segmented electrodes for a directional lead are smaller than typical circumferential or ring electrodes one leads. Therefore, the charge accumulation on the tissue returns through a single small segmented electrode on the direction lead. However, because of their smaller surface area, these smaller electrodes have much higher impedance than the ring electrodes. For example, a segmented electrode on a directional lead may have an impedance of about 2,000 ohms and ring electrode may of have an impedance of about 800 ohms, such that the impedance of the segmented electrode on the direction lead may be about 2.5 times higher than the impedance of a ring electrode. The current flow through the higher impedance segmented electrode is less than the current flow through the lower impedance ring electrode, which lengthens the amount of time to passively recover the charge using the higher impedance segmented electrode. As a result, forcing passive recovery through only the higher impedance, smaller segmented electrode may allow charge to accumulate and cause undesired side effects of the neural anatomy.

### SUMMARY

Various embodiments provide systems and methods to enable a clinician to change the electrodes that are used to passively recover charge. The electrodes may include electrodes that are not activated to deliver the neurostimulation. The electrodes may be chosen to collect unwanted charge from untargeted neural tissue to inhibit the charge from accumulating and causing the undesired side effects. For example, a user input element, such as a slide control, dial or button, on the programmer may be actuated by the clinician to turn on enhanced passive recovery when desired to avoid a side effect. In some embodiments, a clinician may select the anatomical structure (e.g., point and click within an anatomical image) in which it is desired to remove charge accumulation.

An example (e.g., “Example 1”) of a system may include a neurostimulator and a programmer. The neurostimulator may have a plurality of electrodes, and may be configured to deliver neurostimulation by delivering neurostimulation pulses of a first polarity to a neural target using at least one stimulation electrode from the plurality of electrodes and passively recovering charge using a first number of one or more passive electrodes from the plurality of electrodes. The programmer may have a user interface configured to receive a user input for changing a number of passive electrodes used to passively recover charge from the first number to a second number. The programmer may be configured to control the neurostimulator to passively recover charge using the second number of passive electrodes.

In Example 2, the subject matter of Example 1 may optionally be configured such that the plurality of electrodes includes segmented electrodes on a deep brain stimulation (DBS) lead.

In Example 3, the subject matter of any one or more of Examples 1-2 may optionally be configured such that the received user input includes a progressive input to progressively change the number of electrodes.

In Example 4, the subject matter of Example 3 may optionally be configured such that the user interface includes a slider bar configured to be moved to provide the progressive input or a dial configured to be moved to provide the progressive input.

In Example 5, the subject matter of any one or more of Examples 3-4 may optionally be configured such that the user interface is configured to receive an input of a value to provide the progressive input.

In Example 6, the subject matter of any one or more of Examples 1-5 may optionally be configured such that the programmer is configured to automatically control the neurostimulator to passively recover charge using the second number based on the received user input.

In Example 7, the subject matter of any one or more of Examples 1-6 may optionally be configured such that each of the one or more passive electrodes used to passively recover charge is also one of the at least one stimulation electrode used to deliver neurostimulation.

In Example 8, the subject matter of any one or more of Examples 1-6 may optionally be configured such that at least one of the one or more passive electrodes used to passively recover charge is not also used as one of the at least one stimulation electrode used to deliver neurostimulation.

In Example 9, the subject matter of any one or more of Examples 1-8 may optionally be configured such that the user interface is configured to receive a user selection of which of the plurality of electrodes are used to passively recover charge.

In Example 10, the subject matter of any one or more of Examples 1-9 may optionally be configured such that the programmer includes an algorithm configured to determine which of the plurality of electrodes are used to passively recover charge.

In Example 11, the subject matter of Example 10 may optionally be configured such that the programmer is configured to automatically program the neurostimulator with the plurality of electrodes determined by the algorithm to be used to passively recover charge.

In Example 12, the subject matter of Example 10 may optionally be configured such that the programmer is configured to automatically suggest the plurality of electrodes determined by the algorithm to be used to passively recover charge, and the programmer is configured to enable a user to program the neurostimulator with the plurality of electrodes determined by the algorithm to be used to passively recover charge.

In Example 13, the subject matter of any one or more of Examples 10-12 may optionally be configured such that the algorithm is configured to determine which of the plurality of electrodes are used to passively recover charge based on at least one of: image guided information, which of the plurality of electrodes when used to deliver neurostimulation pulses cause a quickest side effect, which of the plurality of electrodes when used to deliver neurostimulation pulses has a smallest therapy window, a location or distance to a stimulation target, a location or distance to side effect tissue, or electrode size.

In Example 14, the subject matter of Example 10 may optionally be configured such that the programmer is configured to receive one or more algorithm inputs used, in addition to the received user input to change the number of passive electrodes, to determine which passive electrodes are used to passively recover charge.

In Example 15, the subject matter of Example 10 may optionally be configured such that the one or more algorithm inputs include at least one of: medical imaging information, a side effect, a clinical effect, a stimulation target, side effect tissue, or lead information.

Example 16 includes subject matter (such as a method, means for performing acts, machine readable medium including instructions that when performed by a machine cause the machine to perform acts, or an apparatus to perform). The subject matter may include delivering neurostimulation using a neurostimulator having a plurality of electrodes, including delivering neurostimulation pulses of a first polarity to a neural target using at least one stimulation electrode from the plurality of electrodes and passively recovering charge using a first number of one or more passive electrodes from the plurality of electrodes, receiving a user input at a user interface of a programmer for changing a number of passive electrodes used to passively recover charge from the first number to a second number, and controlling the neurostimulator, using the programmer, to passively recover charge using the second number of passive electrodes.

In Example 17, the subject matter of Example 16 may optionally be configured such that the plurality of electrodes includes segmented electrodes on a deep brain stimulation (DBS) lead.

In Example 18, the subject matter of any one or more of Examples 16-17 may optionally be configured such that the received user input includes a progressive input to progressively change the number of electrodes.

In Example 19, the subject matter of Example 18 may optionally be configured such that the user interface includes a slider bar configured to be moved to provide the progressive input or a dial configured to be moved to provide the progressive input.

In Example 20, the subject matter of any one or more of Examples 18-19 may optionally be configured such that the user interface is configured to receive an input of a value to provide the progressive input.

In Example 21, the subject matter of any one or more of Examples 16-20 may optionally be configured such that the programmer automatically controls the neurostimulator to passively recover charge using the second number based on the received user input.

In Example 22, the subject matter of any one or more of Examples 16-21 may optionally be configured such that each of the one or more passive electrodes used to passively recover charge is also one of the at least one stimulation electrode used to deliver neurostimulation.

In Example 23, the subject matter of any one or more of Examples 16-21 may optionally be configured such that at least one of the one or more passive electrodes used to passively recover charge is not also used as one of the at least one stimulation electrode used to deliver neurostimulation.

In Example 24, the subject matter of any one or more of Examples 16-23 may optionally be configured to further include receiving a user selection of which of the plurality of electrodes are used to passively recover charge.

In Example 25, the subject matter of any one or more of Examples 16-24 may optionally be configured to further include using an algorithm configured to determine which of the plurality of electrodes are used to passively recover charge.

In Example 26, the subject matter of Example 25 may optionally be configured such that the programmer is configured to automatically program the neurostimulator with the plurality of electrodes determined by the algorithm to be used to passively recover charge.

In Example 27, the subject matter of Example 25 may optionally be configured such that the programmer is configured to automatically suggest the plurality of electrodes determined by the algorithm to be used to passively recover charge, and the programmer is configured to enable a user to program the neurostimulator with the plurality of electrodes determined by the algorithm to be used to passively recover charge.

In Example 28, the subject matter of any one or more of Examples 25-27 may optionally be configured such that the algorithm is configured to determine which of the plurality of electrodes are used to passively recover charge based on at least one of: image guided information, which of the plurality of electrodes when used to deliver neurostimulation pulses cause a quickest side effect, which of the plurality of electrodes when used to deliver neurostimulation pulses has a smallest therapy window, a location or distance to a stimulation target, a location or distance to side effect tissue, or electrode size.

In Example 29, the subject matter of Example 25 may optionally be configured to further include receiving one or more algorithm inputs used, in addition to the received user input to change the number of passive electrodes, to determine which passive electrodes are used to passively recover charge.

In Example 30, the subject matter of Example 29 may optionally be configured such that the one or more algorithm inputs include at least one of: medical imaging information, a side effect, a clinical effect, a stimulation target, side effect tissue, or lead information.

Example 31 includes subject matter that includes non-transitory machine-readable medium including instructions, which when executed by a machine, cause the machine to perform a method for controlling a neurostimulator, having a plurality of electrodes, to deliver neurostimulation including deliver neurostimulation pulses of a first polarity to a neural target using at least one stimulation electrode from the plurality of electrodes and passively recover charge using a first number of one or more passive electrodes from the plurality of electrodes. The method performed by the machine may include receiving a user input at a user interface of a programmer for changing a number of passive electrodes used to passively recover charge from the first number to a second number, and controlling the neurostimulator, using the programmer, to passively recover charge using the second number of passive electrodes. In additional examples, the method performed by the machine in Example 31 may include the method in any of Examples 17-30.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIGS. 8A-8B illustrate examples of an active charge recovery waveform and a passive charge recovery waveform.

FIG. 9 illustrates, by way of example and not limitation, a method for enhancing the passive recovery of charge in a neurostimulation system.

FIG. 10 illustrates, by way of example and not limitation, a system for enhancing the passive recovery of charge in a neurostimulation system.

FIG. 11 illustrates, by way of example and not limitation, a neurostimulation system configured with an electrode activation circuit with digital to analog circuitry (DAC)/passive recovery circuits to provide multiple independent current control (MICC) that provides neurostimulation with passive recovery for select activated electrodes.

FIG. 12 illustrates, by way of example and not limitation, an example of a DAC/passive recovery circuit used in the electrode activation circuit illustrated in FIG. 11.

FIG. 13 illustrates, by way of example and not limitation, a neurostimulation system configured with an electrode activation circuit with DAC circuits to provide multiple independent current control that provides neurostimulation for select activated stimulation electrodes and with passive recovery circuit to provide passive recovery for select activated passive electrodes.

FIG. 14 illustrates, by way of example and not limitation, a DBS lead within neural tissue including a neural target and an avoidance region.

FIG. 15A-15D illustrates by way of example and not limitation, a programmer screen that may be used to implement enhanced recovery.

FIG. 16 illustrates some examples of inputs that may be used for some examples of passive electrode selection.

FIG. 17 illustrates, by way of example and not limitation, a method for initially programming the neurostimulation system using monopolar review to determine clinical effect information and side effect information.

FIG. 18 illustrates, by way of example and not limitation, a method for progressively increasing a number of passive electrodes based on a location of the stimulation electrodes.

FIG. 19 illustrates, by way of example and not limitation, a method for progressively increasing a number of passive electrodes based on a location of an avoidance region where a side effect may occur.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

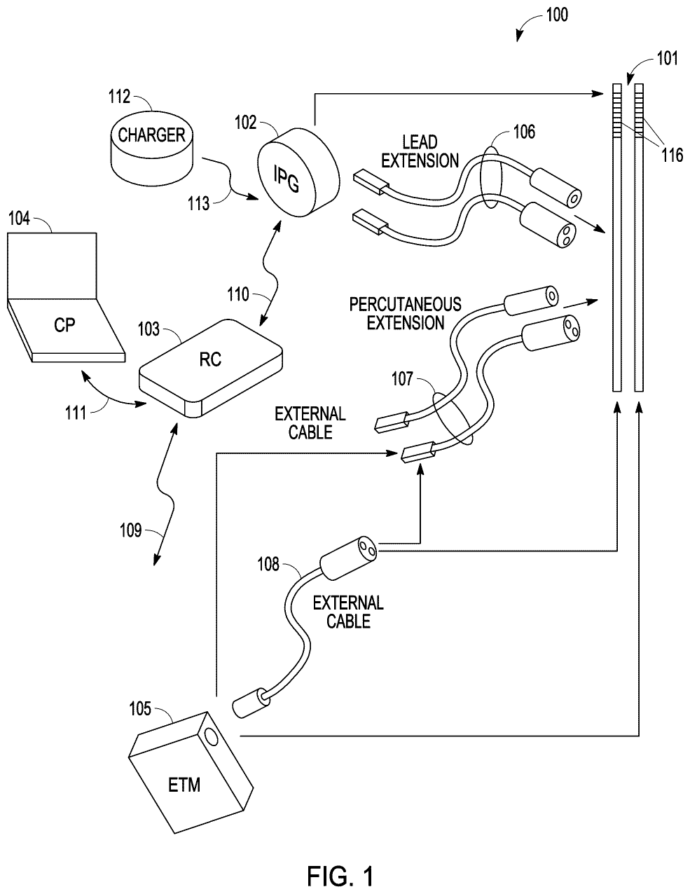

FIG. 1 illustrates, by way of example and not limitation, an electrical stimulation system 100, such as may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters. The leads may be directional leads with a lead marker for use to help determine an orientation for an implanted lead. Directional leads may be used to target neural stimulation for DBS, SCS, PNS or other electrical stimulation.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. The ETM 105 may be used for situations where a brief period of therapy is suitable to achieve the desired effects. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned ON or OFF and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, the IPG 102 may function as programmed without the RC 103 or CP 104 being present. It is noted that some IPGs do not require charging, as some are manufactured with primary batteries with sufficient capacity to provide therapy over a clinically useful duration without recharging.

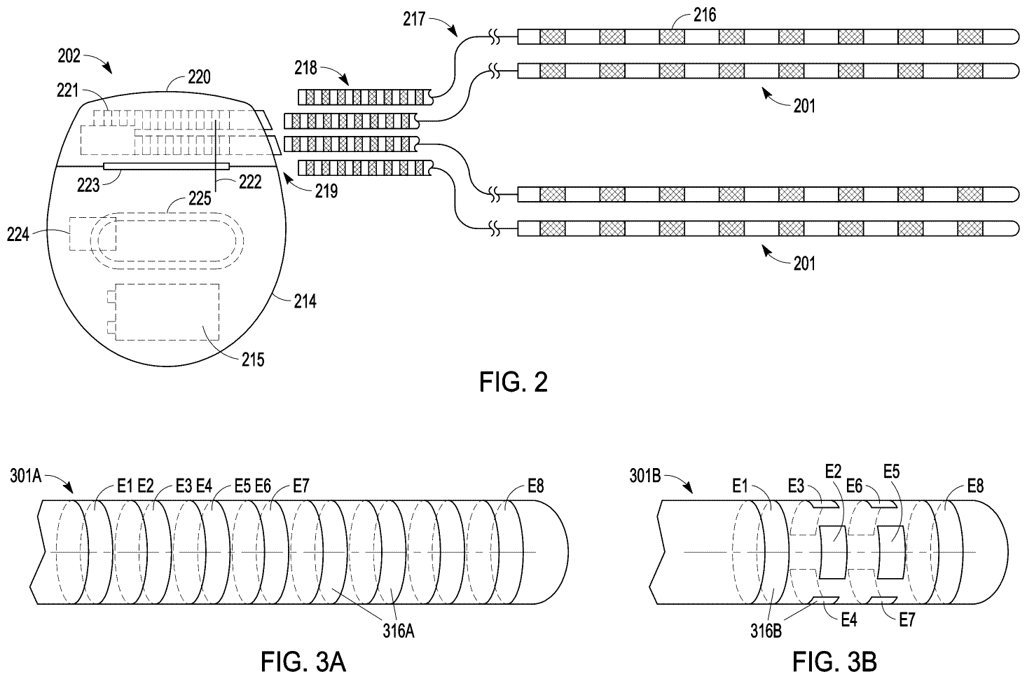

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracting the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some architectures, electrodes of the same polarity can deliver distinct amounts of current simultaneously using multiple electrical sources, to provide greater control of the electric field. In some instances, an electrode might be an anode for a period of time and a cathode for a period of time (e.g., when multiple phases are used, for example, for charge recovery or other purposes). These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering using multiple electrical sources can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length of the lead

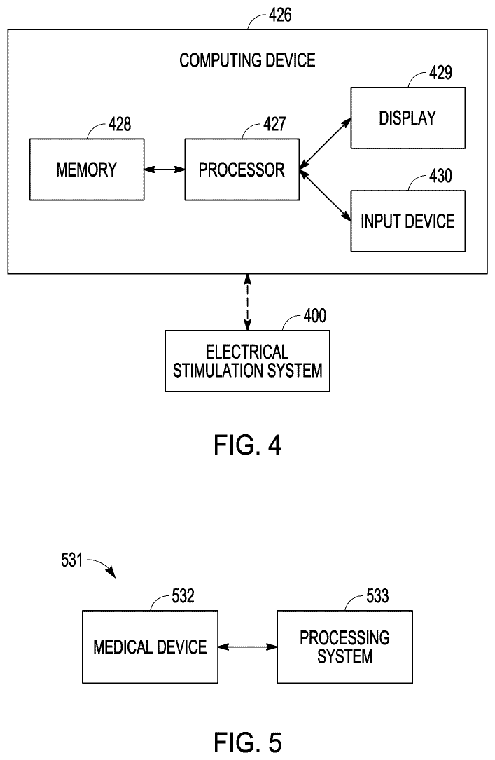

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses. In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein. The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to use at least one directional lead to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device or may be a distributed system across two or more devices including local and/or remote systems. According to various embodiments, the medical system may include at least one medical device configured to treat a condition by delivering a therapy to a patient.

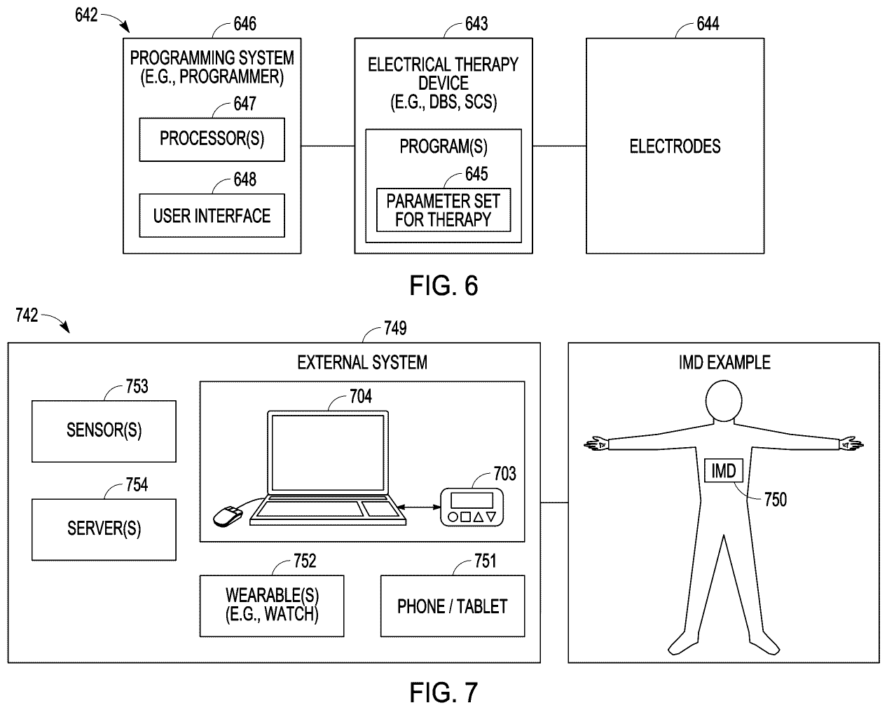

FIG. 6 illustrates, by way of example, an example of an electrical therapy-delivery system. The illustrated system 642 may be a more specific example of the system illustrated in FIG. 5 or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, which may include one or more processors 647 and a user interface 648. The programming system 646 may be used to program and/or evaluate the parameter set(s) used to deliver the therapy. The illustrated system 642 may be a DBS system. In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using an IMD. The IMD may include a DBS stimulator. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote-control device 703, similar to RC 103 in FIG. 1, configured for use by the patient to communicate with and program the neuromodulator. For example, the remote-control device 703 may allow the patient to turn a therapy ON and OFF, change or select programs, adjust patient-programmable parameter(s) of the plurality of modulation parameters, and/or provide inputs used to detect event(s). The patient may use custom external devices to perform specific tasks useful for the event-triggered therapy of that patient. The external devices may communicate directly with the IPG/IMD, or they may communicate with an intermediary device (such as an RC) which in turn communicates with the IPG/IMD. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote-control device 703. The remotely located computer(s) and the IMD 750 may be configured to communicate with each other via another external device such as the programmer 704 or the remote-control device 703. The remote-control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensor(s) 753 and server(s) s 754. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) for the patient and broader patient population(s). The medical record(s) may be stored and accessed using one or more servers (e.g., local or remote servers such as cloud-based servers). The external device may also include device(s) (e.g., app on phone/tablet or a custom device) used by the patient to perform tasks and may also monitor the ability of the patient to perform the task. The external system may be used to process inputs, detect events, analyze the results and/or optimize the training. Processing may be done using cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

Electrical therapy, such as neurostimulation, may be delivered using an electrical waveform between a specified group of electrodes as monophasic electrical energy or multiphasic electrical energy. Monophasic electrical energy takes the form of an electrical pulse train that includes either all negative pulses (cathodic), or alternatively all positive pulses (anodic). Biphasic or multiphasic waveforms alternate between positive and negative polarities.

Multiphasic electrical energy may include a series of biphasic pulses, with each biphasic pulse including a stimulation pulse and a charge recovery pulse. By way of example, the stimulation pulses may be cathodic (negative) during a first phase and the charge recovery pulse may be anodic (positive) during a second phase. The charge recovery may avoid direct current charge transfer through the tissue, which may thereby avoid electrode degradation and cell trauma. That is, charge is conveyed through the electrode-tissue interface via current at an electrode during the stimulation pulse is pulled back off the electrode-tissue interface via an oppositely polarized current at the same electrode during recharge.

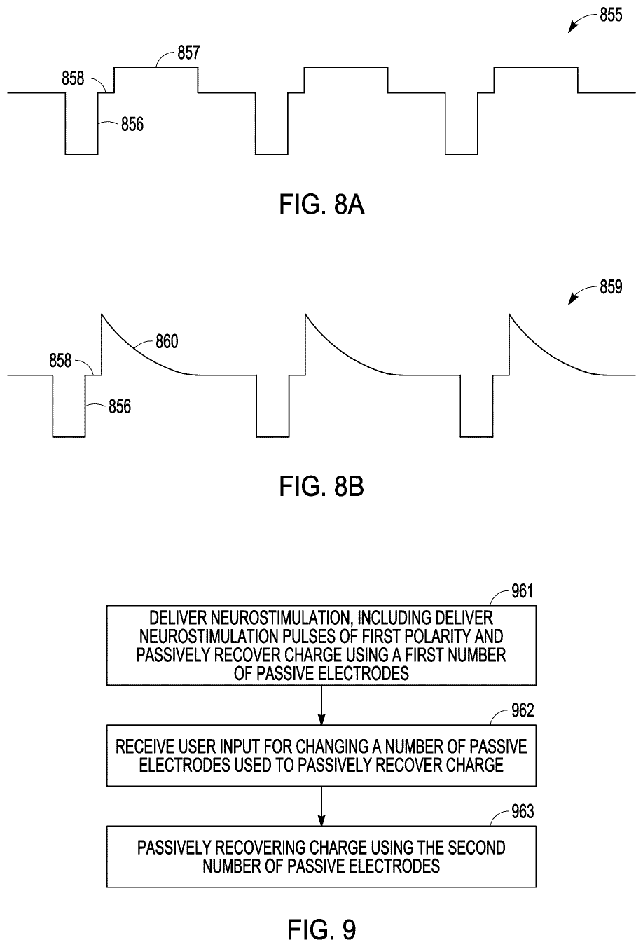

FIGS. 8A-8B illustrate examples of an active charge recovery waveform and a passive charge recovery waveform. The active charge recovery waveform 855 may have a stimulation pulse 856 during the first phase and may have an active charge recovery pulse 857 during the second phase. The active charge recovery pulse may be produced by actively conveying electrical current through the electrode via current or voltage sources. An interphase period 858 may separate the stimulation pulse 856 and the active charge recovery pulse 856. As illustrated in FIG. 8B, the passive charge recovery waveform 859 may have the stimulation pulse 856 in the first phase but the second phase may have a passive charge recovery pulse 860 where electrical current is passively conveyed through the electrode via redistribution of the charge flowing from coupling capacitances present in the circuit. For example, the accumulated charge may flow from the coupling capacitances through resistance to a reference potential. The capacitance and resistance function as an RC discharging circuit causing the passive charge recovery pulse to be an exponentially decaying waveform as illustrated in FIG. 8B. Those of ordinary skill in the electronics art will understand that current represents a rate at which charge flows, and that as the resistance increases, the current or rate of charge flow for discharging the capacitance decreases which increases the amount of time required to discharge the accumulated charge. Although active charge recovery pulses enable faster recharge, the active charge recovery pulses require about twice as much power as a monophasic waveform.

Various embodiments provide systems and methods to enable a clinician to change the electrodes that are used to passively recover charge. Additional electrodes may be selected to increase the overall rate of charge flow. Furthermore, the electrodes may be chosen to collect unwanted charge from untargeted neural tissue to inhibit the charge from accumulating and causing the undesired side effects. For example, a user input element, such as a slide control, dial, button, or value entries on the programmer may be actuated by the clinician to turn on enhanced passive recovery when desired to avoid a side effect. A clinician who is programming DBS and who observes unwanted side effects may be able to gradually introduce an enhanced passive recovery which may include adding electrodes to participate in passive recovery by increasing surface area and lowering the collective impedance. Some embodiments use a progressive input element such as a slide control or dial, to gradually add electrodes used to passively recover charge. In some embodiments, an algorithm may take into consideration which electrodes are already assigned and then gradually include only the adjacent electrodes that have no cathode/anode assignment. Other embodiments may add other electrodes that may not be adjacent to the cathode/anode electrodes. As the slide control continues to move additional unassigned electrodes (e.g., adjacent electrodes) start to participate in the recovery phase of the pulse until the clinician slides it all the way and all electrodes on the lead are used in the recovery phase. Thus, the progressive input element may be used to gradually speed up the charge recovery phase of a neurostimulation pulse by systematically adding electrodes used to passively recover charge, thereby lowering impedance, speeding up the charge recovery, and inhibiting a side effect by avoiding an action potential from taking place in unwanted neuoroanatomy.

A benefit of the enhanced passive recovery is that charge recovery is faster without forcefully adding another stimulation field or another center point of stimulation (CPS) using active recharge. Another benefit of the enhanced passive recovery may include increasing a therapeutic window. For example, if only the electrode used to deliver the stimulation pulse is used to passively recover the charge, the therapeutic effect may be experienced with 2.1 mA of current through the electrode and a side effect may be experienced at 2.5 mA for a therapeutic window of 0.4 mA. The side effect may be caused by charge accumulation because the charge cannot be adequately recovered before the next pulse. However, with enhanced passive recovery, while the therapeutic effect sill may be experienced with 2.1 mA of current through the electrode, the side effect may be experienced at 2.9 mA to provide an increased therapeutic window of 0.8 mA. Larger therapeutic windows provide greater margins for delivering adequate neurostimulation therapy without inducing a side effect.

DBS is provided as an example using enhanced passive charge recovery. Side effect feedback can occur faster than other types of neurostimulation such as, but not limited to, sub-perception threshold SCS. Enhanced passive charge recovery may be used for any type of stimulus. For example, this may be used with super-perception threshold or sub-perception threshold SCS using an array of electrodes on a set of percutaneous leads (e.g., 1-4 leads) or a paddle lead.

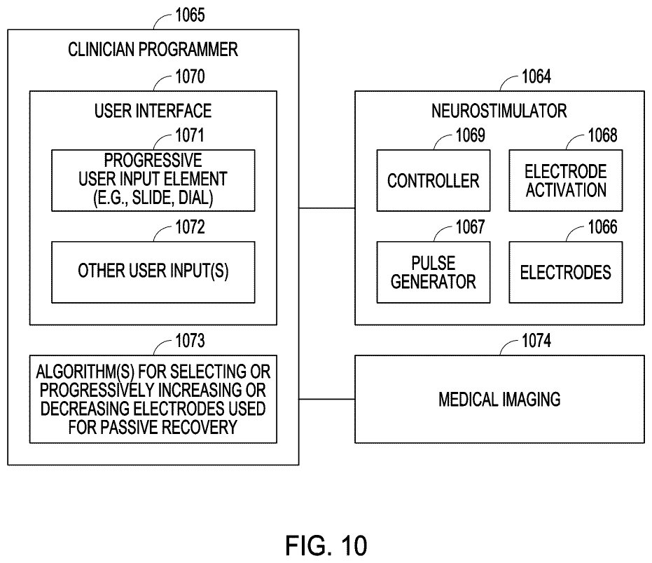

FIG. 9 illustrates, by way of example and not limitation, a method for enhancing the passive recovery of charge in a neurostimulation system. The method may include, at 961, delivering neurostimulation using a neurostimulator having a plurality of electrodes, including delivering neurostimulation pulses of a first polarity to a neural target using at least one stimulation electrode from the plurality of electrodes and passively recovering charge using a first number of one or more passive electrodes from the plurality of electrodes. The electrodes may include segmented electrodes on a deep brain stimulation (DBS) lead. The method may further include, at 962, receiving a user input at a user interface of a programmer for changing a number of passive electrodes used to passively recover charge from the first number to a second number. The received user input may include a progressive input to progressively change the number of electrodes. For example, the user interface may include a slider bar or dial, and the received user input includes movement of the slider bar or movement of the dial to provide the progressive input. In some embodiments, the received user input includes input of a value to provide the progressive input. The method may include, at 963, controlling the neurostimulator, using the programmer, to passively recover charge using the second number of passive electrodes. The programmer may be configured to automatically control the neurostimulator to passively recover charge using the second number based on the received user input.

The method may include receiving a user selection of which of the plurality of electrodes are used to passively recover charge. The method may include using an algorithm to determine which of the plurality of electrodes are used to passively recover charge. The programmer may be configured to automatically program the neurostimulator with the plurality of electrodes determined by the algorithm to be used to passively recover charge and/or may be configured to automatically suggest the plurality of electrodes determined by the algorithm to be used to passively recover charge and enable the user to use the programmer to program the neurostimulator with the plurality of electrodes determined by the algorithm to be used to passively recover charge. The algorithm may be configured to determine which of the plurality of electrodes are used to passively recover charge based on at least one of: image guided information, which of the plurality of electrodes when used to deliver neurostimulation pulses cause a quickest side effect, which of the plurality of electrodes when used to deliver neurostimulation pulses has a smallest therapy window, a location or distance to a stimulation target, a location or distance to side effect tissue, or electrode size. One or more algorithm inputs may be used, in addition to the received user input to change the number of passive electrodes, to determine which passive electrodes are used to passively recover charge. The one or more algorithm inputs may include at least one of: medical imaging information, a side effect, a clinical effect, a stimulation target, side effect tissue, or lead information.

FIG. 10 illustrates, by way of example and not limitation, a system for enhancing the passive recovery of charge in a neurostimulation system. The system may include a neurostimulator 1064 and a programmer 1065 such as a clinician programmer. The neurostimulator 1064 may have a plurality of electrodes 1066. Various embodiments of the neurostimulator and electrodes may be similar to the neurostimulator and electrodes illustrated in at least some FIGS. 1-7.

The illustrated neurostimulator 1064 may include a pulse generator 1067 for generating a neural stimulation waveform (e.g., pulsed waveform), an electrode activation circuit 1068 for controlling the electrodes 1066 used to deliver the neurostimulation pulses and to passively recover charge, and a controller 1069 configured for controlling the pulse generator and the electrode activation. The illustrated programmer 1065 may include a user interface 1070. By way of example, the illustrated user interface 1070 is configured with a progressive user input element 1071 such as a slide or dial that can progressively increase the number of electrodes used to passively recover charge. Similarly, the motion of the progressive user input element may be reversed to passively reduce the number of electrodes used to passively recover charge.

The user interface 1070 may include other user input(s) 1072, which for example, may include inputs for selecting or identifying the neural target to be stimulated, inputs for selecting or identifying avoidance regions which should not be stimulated (e.g., stimulation of the avoidance regions may cause a side effect), inputs for selecting passive electrodes, inputs for identifying when a therapeutic effect (e.g., clinical effect) is experienced, inputs for identifying when a side effect is experienced, or inputs for identifying lead/electrode information such as be used to determine electrode size and/or electrode impedance.

The illustrated programmer 1065 may include one or more algorithms 1073 that may be implemented for selecting or progressively increasing or decreasing a number of electrodes used for passive recovery. The algorithm(s) may be configured to determine which of the plurality of electrodes are used to passively recover charge. In some embodiments, the programmer 1065 may be configured to automatically program the neurostimulator 1064 with the plurality of electrodes determined by the algorithm to be used to passively recover charge. In some embodiments, the programmer 1065 may be configured to automatically suggest the plurality of electrodes determined by the algorithm to be used to passively recover charge, and the programmer may be configured to enable the user to program the neurostimulator with the plurality of electrodes determined by the algorithm to be used to passively recover charge. Some system embodiments may include a medical imaging system 1074 which may provide image guided information capable of being used to determine which of the electrodes are used to passively recover charge.

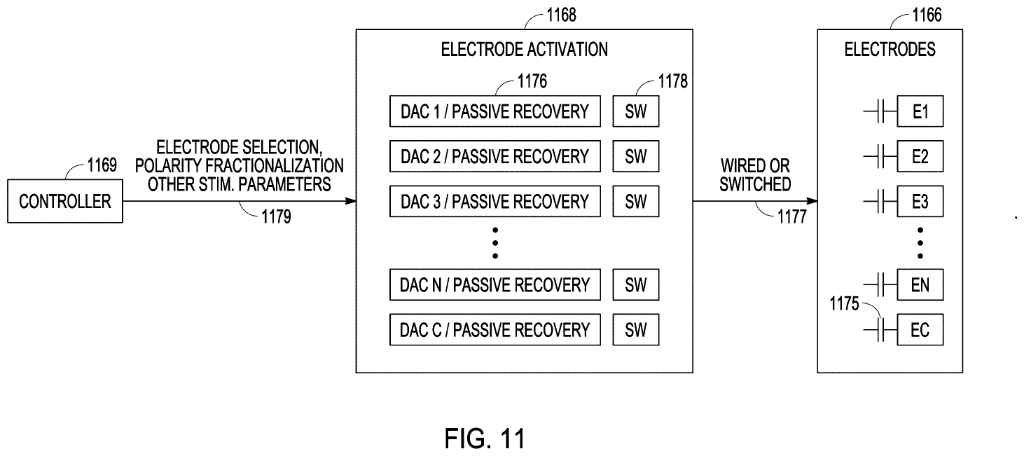

FIG. 11 illustrates, by way of example and not limitation, a neurostimulation system configured with an electrode activation circuit with digital to analog circuitry (DAC)/passive recovery circuits to provide multiple independent current control (MICC) that provides neurostimulation with passive recovery for select activated electrodes. The controller 1169, electrode activation circuit 1168 and electrodes 1166 may be more specific examples of the controller 1069, electrode activation circuit 1068 and electrodes 1066 for the neurostimulator illustrated in FIG. 10. The electrodes 1166 may include N electrodes (E1-EN) on one or more leads and may further include a can electrode (EC) on the housing of the implantable neurostimulator. A capacitor 1175 in series with the electrode prevents direct current (DC) into the tissue. These capacitors may also be referred to as DC-blocking capacitors. The DC-blocking capacitors may prevent a DC current from inadvertently being injected into the tissue if there is a circuit failure.

The electrode activation circuit 1168 may be designed to use multiple independent current control (MICC) technology, which independently controls the current sourced or sunk at each active electrode. The electrode activation circuit 1168 may be configured with both a digital to analog circuit (DAC) and a passive recovery circuit, collectively 1176, for each of the electrodes. Alternatively, there may be a number of DAC/passive recovery circuits 1176 that can be switched into connection with selected electrodes to be activated. Thus, the connection 1177 between the electrode activation circuit 1168 and the electrodes 1166 may either be a wired connection or a switched connection. Each DAC is configured to provide a current source of either a positive polarity (anodic current) or negative polarity (cathodic current), and is capable of controlling the amplitude of the current provided by the current source. The switches 1178 may be used to activate select DAC/passive recovery circuits by, for example, closing a corresponding switch in order to allow pulses generated by the DAC to pass to the activated electrodes and to allow accumulated charge to be passively recovered through the corresponding passive recovery circuit.

The controller 1169 may provide a control signal 1179 to the electrode activation circuit. The control signal may include digital information used to determine electrode selection (selected activation electrodes), polarity (whether the activated electrode functions as a cathode or anode), fractionalization (energy contribution of each cathode toward the overall cathodic energy or energy contribution of each anode toward the overall anodic energy), and other stimulation parameters such as, but not limited to, pulse amplitude, pulse frequency, and pulse width of stimulation pulses.

In the illustrated embodiment, only those electrodes 1166 that are actively used to deliver stimulation pulses either as an anode or cathode may be used to passively recover charge. Therefore, in the illustrated example, an electrode is added to passively recover charge by activating the electrode to deliver charge at a low fractionalization value (e.g., 1%).

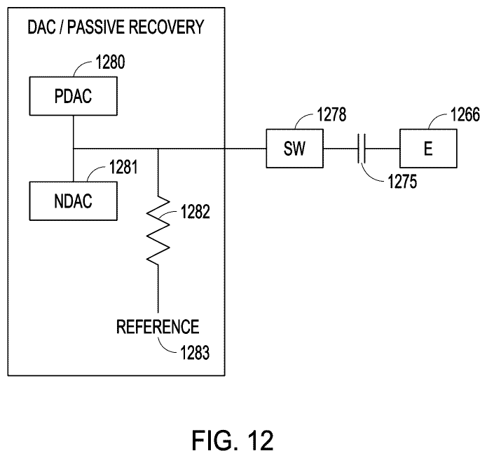

FIG. 12 illustrates, by way of example and not limitation, an example of a DAC/passive recovery circuit 1276, such as the DAC/passive recovery circuit 1176 used in the electrode activation circuit illustrated in FIG. 11. Each of the DAC/passive recovery circuits 1276 may include a PDAC 1280 configured to provide a positive current source and an NDAC 1281 configured to provide a negative current source. The PDAC 1280/NDAC 1281 combination may be connected to the electrode 1266 via switch 1278 for use in delivering the stimulation pulse during a first phase. During the second phase, accumulated charge may be discharged through a resistor 1282 to a reference potential 1283. The capacitor 1275 and resistor function as an RC discharging circuit causing the passive charge recovery pulse to be an exponentially decaying waveform (see, for example, FIG. 8B).

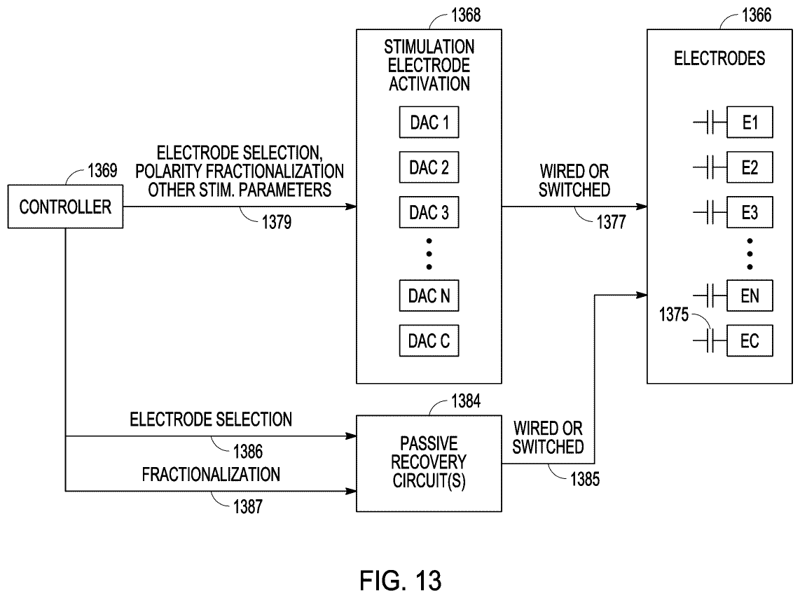

FIG. 13 illustrates, by way of example and not limitation, a neurostimulation system configured with an electrode activation circuit with DAC circuits to provide multiple independent current control that provides neurostimulation for select activated stimulation electrodes and with passive recovery circuit to provide passive recovery for select activated passive electrodes. The controller 1369, electrode activation circuit 1368 and electrodes 1366 may be more specific examples of the controller 1069, electrode activation circuit 1068 and electrodes 1066 for the neurostimulator illustrated in FIG. 10. The electrodes 1366 may include N electrodes (E1-EN) on one or more leads and may further include a can electrode (EC) on the housing of the implantable neurostimulator. Similar to FIG. 11, a DC-blocking capacitor 1375 in series with the electrode prevents direct current (DC) into the tissue. These capacitors may also be referred to as DC-blocking capacitors.

In the illustrated neurostimulation system of FIG. 13, a stimulation electrode activation circuit 1368 and a passive recovery circuit 1384 may be separately controlled. Thus, the controller 1369 may control the electrodes used to deliver the stimulation pulse in the first phase of the waveform, and may separately control the electrodes used to passively recover charge in the second phase of the waveform. Each electrode may have its own, dedicated DAC (e.g., DAC 1-DAC N and DAC C) and/or its own dedicated passive recovery circuit such that the connection 1277 between the electrodes 1366 and the stimulation electrode activation circuit 1368 and the connection 1385 between the electrodes 1366 and the passive recovery circuits 1384 may be wired connections. In other embodiments, there may be fewer stimulation electrode activation circuits 1368 than electrodes 1366 such that the connection 1377 from the stimulation electrode activation circuits to the electrodes may be switched connections to the selected activated electrodes and/or there may be fewer passive recovery circuits 1384 than electrodes 1366 such that that the connection 1385 from the passive recovery circuits 1384 to the electrodes 1366 may be switched connections to the selected electrodes. The controller 1369 may control the stimulation activation circuit 1368 using a digital control signal 1379 that identifies the electrode selection for the activated electrodes, the polarity of individual ones of the activated electrodes, the fractionalization (energy contribution) for individual ones of the activated electrodes and other stimulation parameter such as amplitude, pulse frequency and pulse width. The controller 1369 may similarly control the passive recovery circuit 1384 using a digital control signal 1386 that identifies the electrode selection for passive recovery. Some embodiments may also provide fractionalization information 1387, which may be part of digital signal 1386, for individual ones of the selected electrodes to control the contribution of each passive electrode toward recovering charge. For example, variable resistances may be used to control the amount of current flowing from each of the selected passive recovery electrodes. This fractionalization of passive recovery electrodes may be used to prioritize where the charge is most quickly removed from the tissue.

Some embodiments may stagger times when passive electrodes are activated for passive recovery. For example, passive recovery may be turned on for selected electrodes near side effect structures prior to the normal interphase expiration, while passive recovery may be turned on for the other electrodes that were involved in delivering the charge in order to allow charge to be recovered from tissue earlier in areas that might cause a side effect. Some embodiments provide a “static” passive recovery using a passive electrode directly over the avoidance region. This may be constantly on to always removed unwanted charge from accumulating at the avoidance region.

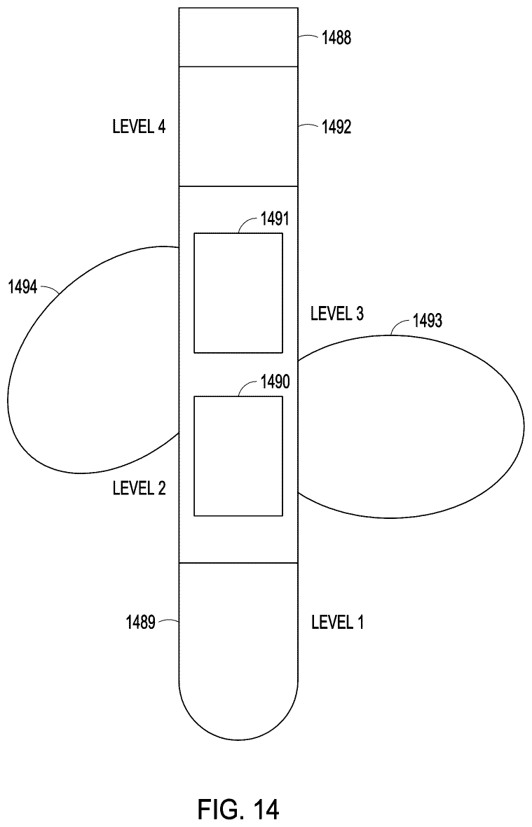

FIG. 14 illustrates, by way of example and not limitation, a DBS lead within neural tissue including a neural target and an avoidance region. The illustrated DBS lead 1488 may include four levels of electrodes. The first or distal level may be a tip electrode 1489 that extends completely around the periphery of the lead. The second and third levels may include electrode segments 1490, 1491 for directional control. Each electrode segment may extend only around a portion of the periphery of the lead. For example, each of the second and third levels may have three electrode segments. The fourth or proximal electrode level may be a ring electrode 1492 that extends completely around a periphery of the lead. The lead is implanted in tissue. For example, the tissue may be, but is not limited to, brain tissue. There may be one or more targeted region(s) 1493 in the tissue that are targeted for stimulation. For example, there may be more than one stimulation channel that can be independently controlled to target one of the targeted region(s). There also may be one or more avoidance regions 1494 within the tissue. The electrode selection and fractionalization may be selected to appropriately generate an electric field to stimulate the targeted region(s) while not stimulating the avoidance region(s). Furthermore, the electrodes for passively recovering charge may be selected to preferentially remove charge quickly from tissue near the avoidance region(s).

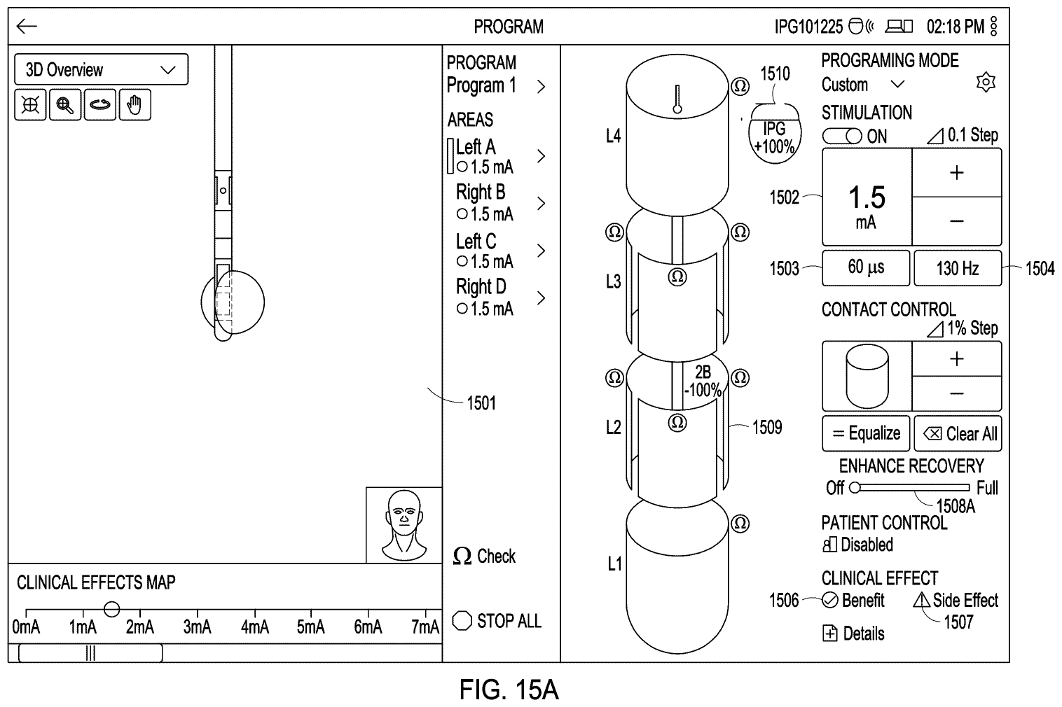

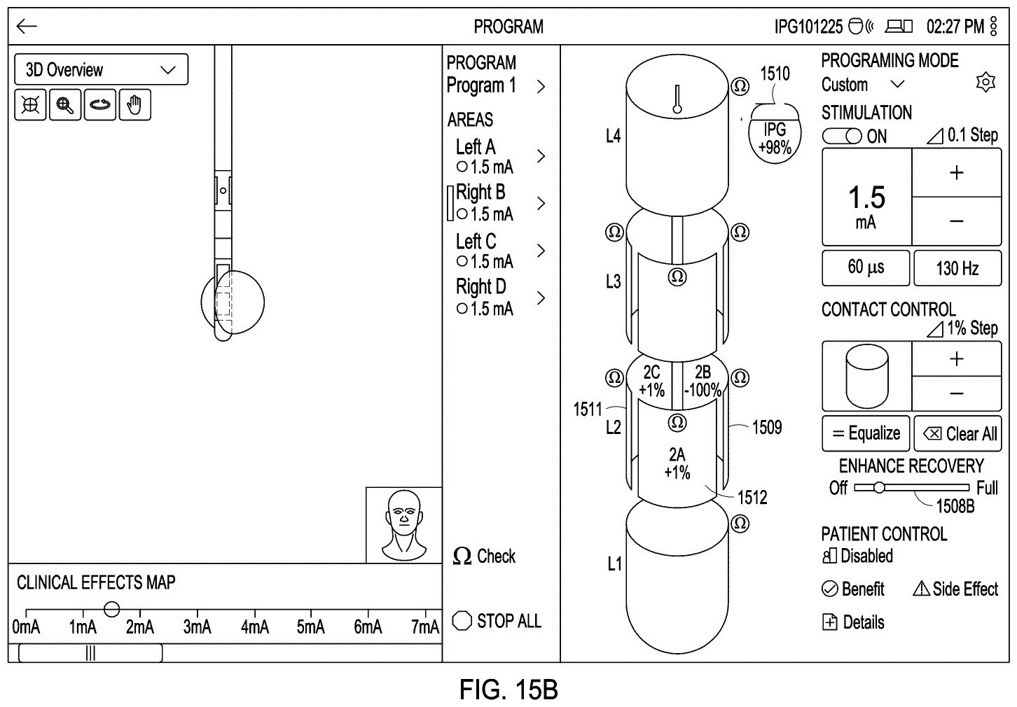

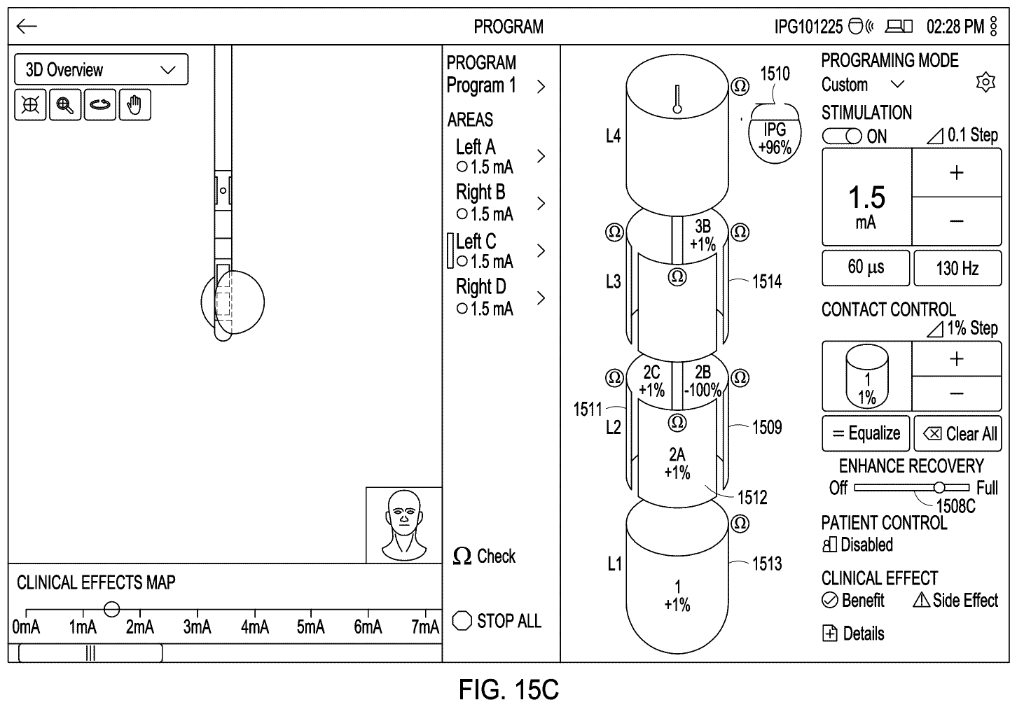

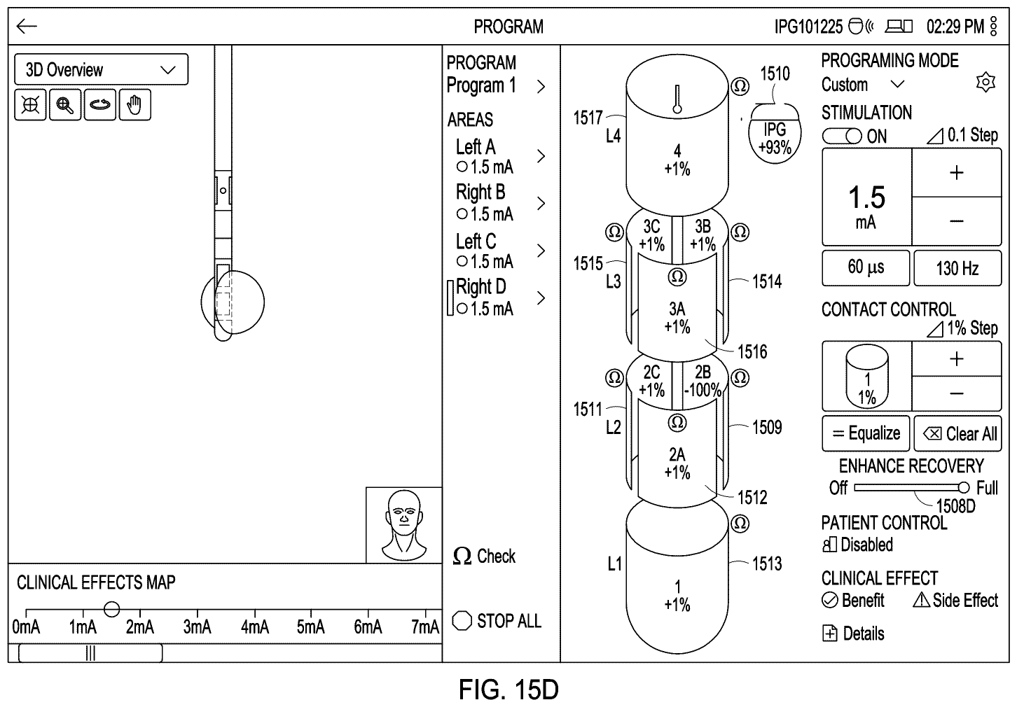

FIG. 15A-15D illustrates by way of example and not limitation, a programmer screen that may be used to implement enhanced recovery. The illustrated programmer screen may be used with a neurostimulation system design of FIG. 11 where passive recovery only happens using stimulation electrodes that have been activated. The left side of the screen shows a clinical effects map 1501 that generally illustrates a region of stimulation with respect to the lead. The screen may include portion, such as illustrated toward the top right, used to control the amplitude 1502 (e.g., 1.5 mA), pulse width 1503 (e.g., 60 μs), and pulse frequency 1504 (e.g., 130 Hz), a representation of the electrodes on the lead as well as the activated electrodes to deliver the neural stimulation. The screen may include a portion, such as illustrated toward the bottom right, used to by the user to indicate when tested stimulation is therapeutic by providing a benefit 1506, when tested stimulation provides a side effect 1507, and other details. This portion of the screen also may include a progressive user element such as a slider bar 1508A used to progressively control the enhanced passive recovery. The stimulation is illustrated as being delivered between one of the segmented electrodes 1509 in the second level and the can electrode 1510. The entire (100%) of the cathodic energy is delivered using electrode 1509 and 100% of the anodic energy is provided by the can electrode 1510 on the IPG. In FIG. 15A, the enhanced passive recovery is “OFF”. Thus, only the activated can electrode 1510 and the activated electrode segment 1509 are used to recover charge.

FIG. 15B illustrates, by way of example and not limitation, a programmer screen where enhanced recovery is on and two electrodes are added for passively recovering charge. The enhanced recover slider bar 1508B is used to progressively add passive electrodes. In the example illustrated in FIG. 15B, the slider bar 1508B is at about 25% and the two additional segment electrodes 1511 and 1512 in the second level are added. The entire (100%) of the cathodic energy continues to be delivered using electrode 1509. However, the anodic energy is fractionalized among the can electrode 1510 at 98%, electrode segment 1511 at 1% and electrode segment 1512 at 1%. The 1% contribution of each of electrode segments 1511 and 1512 is very small and does not significantly affect the neurostimulation field. However, by being activated, these electrode segments 1511 and 1512 are used along with the can electrode 1510 and electrode segment 1509 to passively recover charge. The overall area of the passive electrodes increases, which lowers the effective impedance to the reference potential and increases the speed of the charge recovery.

FIG. 15C illustrates, by way of example and not limitation, a programmer screen where enhanced recovery is on and four electrodes are added for passively recovering charge. In the example illustrated in FIG. 15C, the slider bar 1508C is at about 75% and both the tip electrode 1513 at the first level and segment electrode 1514 in the third level are added. The entire (100%) of the cathodic energy continues to be delivered using electrode 1509. However, the anodic energy is fractionalized among the can electrode at 96%, electrode segment 1511 at 1%, electrode segment 1512 at 1%, electrode segment 1514 at 1% and electrode tip 1513 at 1%. The 1% contributions are very small and do not significantly affect the neurostimulation field. However, by being activated, these electrode segments 1511, 1512, and 1514, and tip electrode 1513 are used along with the can electrode 1510 and electrode segment 1509 to passively recover charge. The overall area of the passive electrodes continues to increase, which continues to lower the effective impedance to the reference potential and speed up the charge recovery.

FIG. 15D illustrates, by way of example and not limitation, a programmer screen where enhanced recovery is on and all available electrodes are added for passively recovering charge. In the example illustrated in FIG. 15D, the slider bar 1508D is completely ON (100%) and all of the available electrodes are used to passively recover charge. The entire (100%) of the cathodic energy continues to be delivered using electrode 1509. However, the anodic energy is fractionalized among the can electrode 1510 at 93%, electrode segment 1511 at 1%, electrode segment 1512 at 1%, tip electrode 1513 at 1%, electrode segment 1514 at 1%, electrode segment 1515 at 1%, electrode segment 1516 at 1% and ring electrode 1517 at 1%. The 1% contributions are very small and do not significantly affect the neurostimulation field. However, by being activated, all of these electrodes are used to passively recover charge. The overall area of the passive electrodes continues to increase, which continues to lower the effective impedance to the reference potential and speed up the charge recovery.

In the embodiment illustrated in FIGS. 15A-15C, the order in which the electrodes are added for use to passively recover charge generally corresponds to adding the electrodes closed to the activated stimulation first. However, algorithms may add passive electrodes in other orders based on various algorithm inputs. It is also expressly noted that the starting or initial set of passive electrodes may vary in location, and/or may vary in the number of passive electrodes used as a default. Some embodiments allow the user to program or set the initial set or initial number (including percentage) of electrodes used in the algorithm(s).

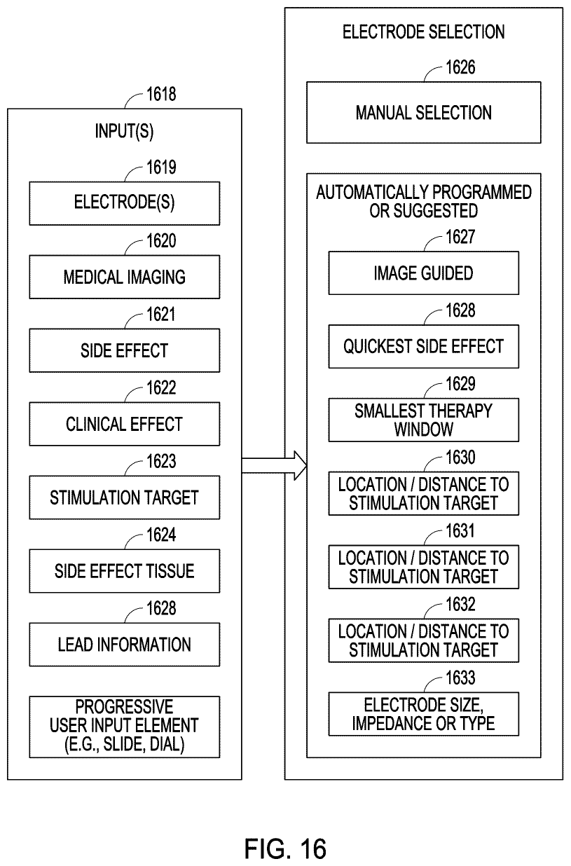

FIG. 16 illustrates some examples of inputs that may be used for some examples of passive electrode selection. An example of an algorithm input 1618 is a selection of electrode(s) 1619 to be used to passively recover charge. This example may be favored by some clinicians who may know where accumulated charge needs to be removed. For example, they may desire to remove charge from the subthalamic nucleus (STN) brain region. In another example, they may desire to deliver electrical therapy to the STN brain region (or other targeted region) and then quickly remove the charge from the STN brain region (or the other targeted region). In some embodiments, the user is able to set a default set of passive electrodes to be used and/or is able to set a default order for adding passive electrodes. Another example of an algorithm input 1618 is medical imaging 1620 information. The visualization and structural assignment (including side effect assignment(s)) inputs from medical imaging software may be inputted into the algorithm. This may be used, for example, to support the automatic detection and prevention of possible side effect. The programmer may be configured to automatically assign those electrodes closest to the identified side effect structures as passive recovery electrodes, or the programmer may suggest the electrodes to be assigned as passive recovery electrodes. Other examples of algorithm inputs 1618 are selection(s) or identification(s) of a side effect 1621 and/or a clinical effect 1622. This may be identified during a monopolar review or other initial programming process where different stimulation electrodes are tested for clinical effects and/or side effects. In some embodiments, the user is able to select the input(s) that they want to be used by the algorithm to select the initial passive electrodes as well as the progression of how passive electrodes are added or removed. Other examples of algorithm inputs 1618 are selection(s) or identification(s) of a stimulation target 1623 and/or side effect tissue (also referred to as avoidance regions) 1624. This may be identified by selection within an image of the tissue or other selection. The selections may be identified using structural assignments from medical imaging. Another example of an algorithm input 1618 is lead information 1625. For example, the particulars of an electrode array may be acquired using a lead identifier. Thus, the electrode arrangements, sizes of individual electrodes, impedances of individual electrodes, and the like may be determined using the lead identifier.

The algorithm(s) may use one or more of these algorithm inputs 1618, along with the user input element (e.g., slider bar) to add or remove passive electrodes, to suggest or automatically assign passive electrodes. For example, the user may simply manually select the passive electrodes 1626. In other examples, the programmer is configured to use the algorithm(s) to automatically suggest the electrodes to be used as passive electrodes or automatically program the neuromodulator with the electrodes to be used passive electrodes. Some embodiments of the programmer allow the user to select one or more of the criteria to be used by the algorithm to select the passive electrodes. For example, the selection may be based on one or more of an image guided information 1627, a quickest side effect 1628, a smallest therapy window 1629, the stimulation electrode(s) used to deliver the stimulation pulse 1630, a location or distance to a stimulation target 1631, a location or distance to side effect tissue and electrode size 1632, impedance or type (e.g., tip electrode, ring electrode, segmented electrode, electrode on another lead, and the like) 1633. Some more advanced embodiments may weight the criteria used by the algorithm. The weights may be inputted by the user or may be developed using machine learning algorithms.

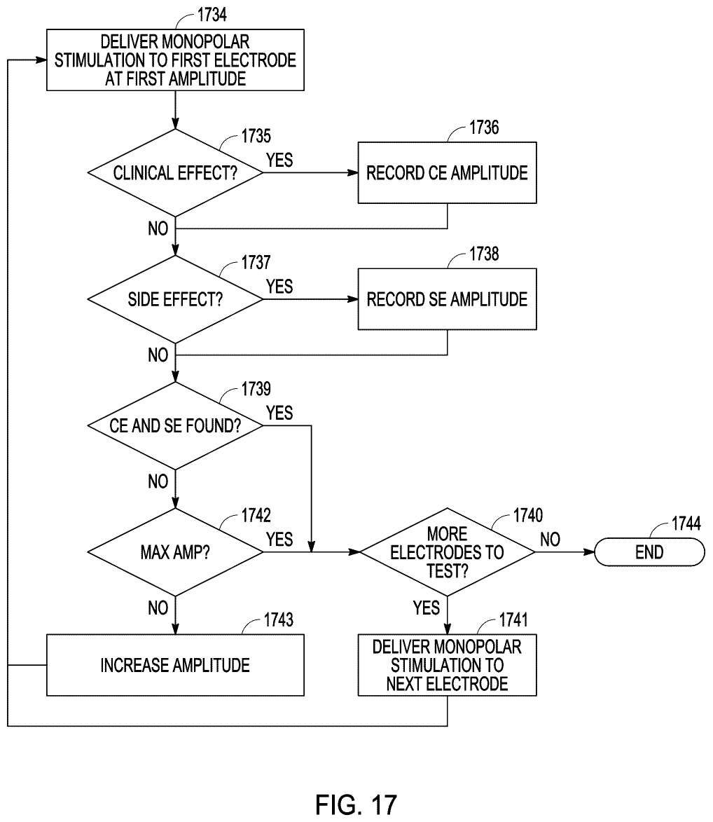

FIG. 17 illustrates, by way of example and not limitation, a method for initially programming the neurostimulation system using monopolar review to determine clinical effect information and side effect information. Monopolar review tests one electrode at a time using 100% of the stimulation energy. It is noted that more than one electrode may be tested at a time. For example, the available electrodes may be divided into two groups and each group of electrodes may be tested simultaneously to quickly identify where the stimulation target is located to identify the group as candidate electrodes. The process may repeat until the stimulation electrode(s) are identified to stimulate the stimulation target. In contrast, the monopolar review tests the available electrodes one electrode at a time. Monopolar stimulation may be delivered to a first electrode at a first amplitude 1734. If there is a clinical effect (e.g., effective therapy) as determined at 1735, the amplitude is recorded as the clinical effect (CE) amplitude 1736. If there is a side effect as determined at 1737, the amplitude is recorded as the side effect (SE) amplitude 1738. If both the CE amplitude and the SE amplitude have been found as determined at 1739, then the process may proceed to determine if there are more electrodes to test 1740. If there are, the process delivers monopolar stimulation to the next electrode at the first amplitude 1741 and returns to 1735 to check for a clinical effect and side effect. If both the CE amplitude and the SE amplitude have not been found as determined at 1739, and if the maximum amplitude has not been delivered as determined at 1742, then the method may increase the amplitude of the stimulation to be delivered to the first electrode 1743 and return to 1735 to check for a clinical effect and side effect. However, if the maximum amplitude has been delivered for the tested electrode as determined at 1742, the process may proceed to determine if there are more electrodes to test 1740. If there are, the process delivers monopolar stimulation to the next electrode at the first amplitude 1741 and returns to 1735 to check for a clinical effect and side effect. After there are no more electrodes to test, as determined at 1740, the process may end 1744.

This process may be used to identify the clinical effect, the side effect, and the therapy window. Some embodiments may vary. For example, a system may be designed such that, if the system determines that the clinical effect amplitudes continue to trend higher or become undetectable, then the process may determine not to test the remaining number of electrodes.

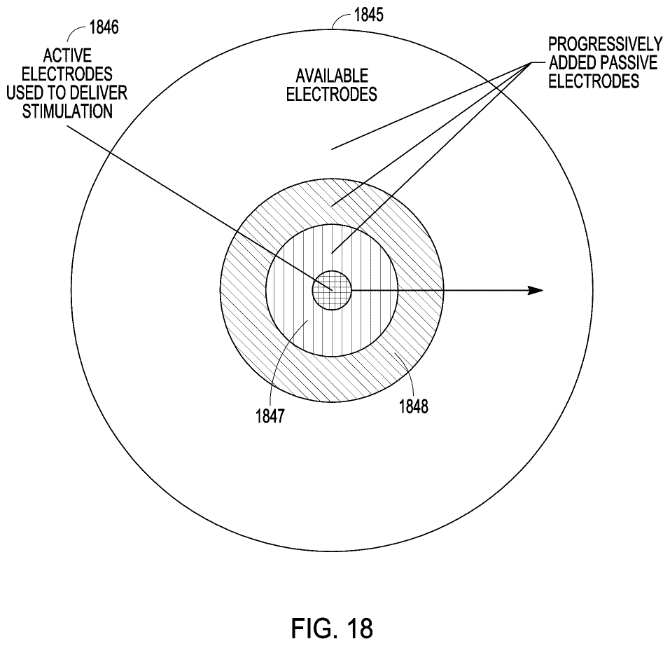

FIG. 18 illustrates, by way of example and not limitation, a method for progressively increasing a number of passive electrodes based on a location of the stimulation electrodes. It is noted that this illustration does not specifically represent any particular electrode arrangement or any particular lead. Rather, the large circle 1845 represents a set of all electrodes that are available for selection to be passive electrodes and/or active stimulation electrodes. A number of electrodes are selected as active electrodes 1846 used to deliver the stimulation pulse (e.g., first phase of the stimulation waveform). These electrodes also may be selected as passive electrodes for the passive recovery (e.g., second phase of the stimulation waveform). As the progressive user element increases, the system may add additional electrodes 1847 closest to the active electrode first, and then progressively add additional passive electrodes 1848 further away from the active electrodes 1846 as the progressive user element continues to increase. Thus, by way of example and not limitation, a clinician may desire to deliver electrical therapy to a targeted region (e.g., STN brain region or other targeted region) and then may use enhanced passive recovery to more quickly remove the charge from the targeted region (e.g., the STN brain region or the other targeted region).

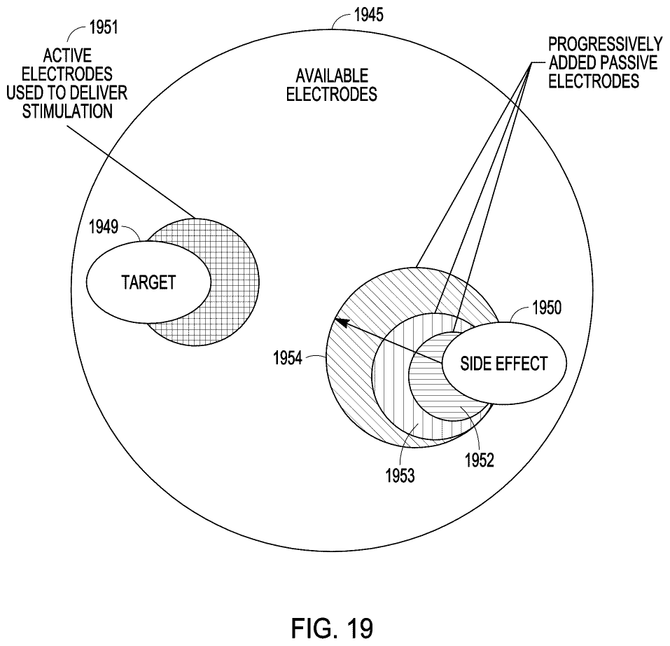

FIG. 19 illustrates, by way of example and not limitation, a method for progressively increasing a number of passive electrodes based on a location of an avoidance region where a side effect may occur. It is again noted that this illustration does not specifically represent any particular electrode arrangement or any particular lead. The large circle 1945 represents a set of all electrodes that are available for selection to be passive electrodes and/or active stimulation electrodes. Also illustrated is a neural stimulation target 1949 and a side effect or avoidance region 1950. A number of electrodes are selected as active stimulation electrodes 1951 used to deliver the stimulation pulse (e.g., first phase of the stimulation waveform). The initial passive electrodes 1952 for the passive recovery pulse (e.g., second phase of the stimulation waveform) may be selected to be other electrodes, distinct from the active stimulation electrodes 1951, near the side effect region. As the progressive user element increases, the system may add additional electrodes 1953 between the side effect region 1950 and the stimulation target 1949 closest to the side effect region 1950, and then progressively add additional passive electrodes 1954 further away from the side effect region 1950 as the progressive user element continues to increase. Thus, by way of example and not limitation, a clinician may desire to deliver electrical therapy to a targeted brain region other than the STN brain region but the clinician may desire to remove charge from the STN brain region (or other non-targeted region) to avoid undesired effects of charge accumulation at the STN brain region (or the other non-targeted region).

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering neurostimulation using a neurostimulator having a plurality of electrodes, including delivering neurostimulation pulses of a first polarity to a neural target using at least one stimulation electrode from the plurality of electrodes and passively recovering charge using a first number of one or more passive electrodes from the plurality of electrodes;
receiving a user input at a user interface of a programmer for changing a number of passive electrodes used to passively recover charge from the first number to a second number; and
controlling the neurostimulator, using the programmer, to passively recover charge using the second number of passive electrodes.

2. The method of claim 1, wherein the plurality of electrodes includes segmented electrodes on a deep brain stimulation (DBS) lead.

3. The method of claim 1, wherein the received user input includes a progressive input to progressively change the number of electrodes.

4. The method of claim 3, wherein the user interface includes a slider bar or dial, and the received user input includes movement of the slider bar or movement of the dial to provide the progressive input.

5. The method of claim 3, wherein the received user input includes input of a value to provide the progressive input.

6. The method of claim 1, wherein the programmer automatically controls the neurostimulator to passively recover charge using the second number based on the received user input.

7. The method of claim 1, wherein each of the one or more passive electrodes used to passively recover charge is also one of the at least one stimulation electrode used to deliver neurostimulation.

8. The method of claim 1, wherein at least one of the one or more passive electrodes used to passively recover charge is not also used as one of the at least one stimulation electrode used to deliver neurostimulation.

9. The method of claim 1, further comprising receiving a user selection of which of the plurality of electrodes are used to passively recover charge.

10. The method of claim 1, further comprising using an algorithm to determine which of the plurality of electrodes are used to passively recover charge.

11. The method of claim 10, wherein the programmer is configured to automatically program the neurostimulator with the plurality of electrodes determined by the algorithm to be used to passively recover charge.

12. The method of claim 10, wherein the programmer is configured to automatically suggest the plurality of electrodes determined by the algorithm to be used to passively recover charge, and enable a user to use the programmer to program the neurostimulator with the plurality of electrodes determined by the algorithm to be used to passively recover charge.

13. The method of claim 10, wherein the algorithm is configured to determine which of the plurality of electrodes are used to passively recover charge based on at least one of: image guided information, which of the plurality of electrodes when used to deliver neurostimulation pulses cause a quickest side effect, which of the plurality of electrodes when used to deliver neurostimulation pulses has a smallest therapy window, a location or distance to a stimulation target, a location or distance to side effect tissue, or electrode size.

14. The method of claim 10, further comprising receiving one or more algorithm inputs used, in addition to the received user input to change the number of passive electrodes, to determine which passive electrodes are used to passively recover charge.

15. The method of claim 14, wherein the one or more algorithm inputs include at least one of: medical imaging information, a side effect, a clinical effect, a stimulation target, side effect tissue, or lead information.

16. A non-transitory machine-readable medium including instructions, which when executed by a machine, cause the machine to perform a method for controlling a neurostimulator, having a plurality of electrodes, to deliver neurostimulation including deliver neurostimulation pulses of a first polarity to a neural target using at least one stimulation electrode from the plurality of electrodes and passively recover charge using a first number of one or more passive electrodes from the plurality of electrodes, the method performed by the machine executing the instructions including:
receiving a user input at a user interface of a programmer for changing a number of passive electrodes used to passively recover charge from the first number to a second number; and
controlling the neurostimulator, using the programmer, to passively recover charge using the second number of passive electrodes.

17. A system, comprising:
a neurostimulator having a plurality of electrodes, wherein the neurostimulator is configured to deliver neurostimulation by delivering neurostimulation pulses of a first polarity to a neural target using at least one stimulation electrode from the plurality of electrodes and passively recovering charge using a first number of one or more passive electrodes from the plurality of electrodes; and
a programmer having a user interface configured to receive a user input for changing a number of passive electrodes used to passively recover charge from the first number to a second number, wherein the programmer is configured to control the neurostimulator to passively recover charge using the second number of passive electrodes.

18. The system of claim 17, wherein the plurality of electrodes includes segmented electrodes on a deep brain stimulation (DBS) lead.

19. The system of claim 17, wherein the received user input includes a progressive input to progressively change the number of electrodes.

20. The system of claim 17, wherein the programmer is configured to automatically control the neurostimulator to passively recover charge using the second number based on the received user input.
